(19) United States
(12) Patent Application Publication
(10) Pub. No.: US 2006/0128945 A1
(43) Pub. Date: Jun. 15, 2006

(54) Reaction process

(76) Inventor: Conan J. Fee, Hamilton (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/526,260

(22) PCT Filed: Sep. 1, 2003
(86) PCT No.: PCT/NZ03/00194

§ 371 Date: Dec. 22, 2005

(30) Foreign Application Priority Data
Sep. 1, 2002 (NZ) 519011

(51) Int. Cl. C07K 14/47; C08G 63/91

(52) U.S. Cl. 530/350; 525/54.1

(57) ABSTRACT

A reaction process, characterised by the step of introducing reaction components to a medium, wherein at least one of the reaction components has a different flow rate from the other reaction component(s) through the medium, so that a moving reaction phase is formed which causes reaction products.

### TECHNICAL FIELD

This invention relates to a reaction process.

Specifically, this invention relates to a technique for controlling chemical reactions and enabling the separation of reaction products.

### BACKGROUND ART

A chemical reaction may be defined as a process in which one or more substances (reaction components) are changed chemically into one or more different substances (products).

While simple chemical reactions can be controlled by conditions such as temperature, or the concentration of one or more of the reaction components and/or products, many reactions are controlled by conditions based on complex interactions between reaction components, catalysts and/or active sites of the reaction components.

In reactions where two or more substances combine to form a single substance, the presence of multiple active sites can result in the formation of a range of different products.

To produce a given substance, such reactions may need to be controlled to attempt to limit the range of products formed and thus reduce the requirement for lengthy separation and purification steps. Such techniques are well known in the art and it is anticipated these would be known to a skilled addressee.

However, these techniques are typically difficult to control and often result in little differences to the degree of product heterogeneity.

A number of separation techniques are widely used for the separation of products from biological or chemical reactions.

Typically, these techniques are based on the physicochemical properties of the compounds in question. Separation may occur based on differences in solubility, charge, absorption or molecular size of the compounds of interest.

Chromatographic separation covers a multitude of these techniques, for example liquid chromatography may be defined as a liquid mobile phase passing over or through a solid or gel stationary phase, with or without the application of voltage.

The choice of an appropriate separation technique depends on a number of factors, including the reasons for the analysis, the amount of sample available, the need to preserve the activity or function of the sample, the desired purity, the equipment available, the type of compounds present and the cost of different techniques.

Typically, large scale methods are used for crude isolations of large quantities of compounds, whereas small scale methods are available for compounds which are expensive or are available only in small quantities.

One common method of separating compounds is done according to their molecular weight. Size exclusion chromatography, sometimes known as gel filtration, is one method used to separate compounds according to their size. This technique uses a column packed with porous beads.

Molecules larger than the pores move quickly through the column, whereas smaller molecules enter the pores in the beads and become entrained, and thus move slowly through the column. By this method, molecules are eluted off the column in order of decreasing size.

While molecular weights of unknown compounds can be determined by comparing their elution volumes to that of known reference samples, one problem with this method is that molecular weights in many cases cannot be directly related to elution times for different shaped molecules.

In practice, separation depends on the Stokes radius of a compound, rather than directly on its molecular weight. The Stokes radius is the average radius that a compound has in solution, depending on its 3-dimensional structure rather than directly on its size.

One example of this are PEGylated compounds.

Small therapeutic proteins have a short half life in-vivo due to filtration and excretion through the kidneys. Their circulation half-life can be dramatically improved by increasing their molecular size and shielding the molecules from degradation through the attachment of one or more polyethylene glycol (PEG) groups.

In standard “liquid-phase” protein PEGylation methods, native protein is mixed with activated PEG for a specified time. Generally the reaction temperature and pH controls the reaction rate.

Mono-PEGylated protein molecules are formed initially, followed by subsequent residual active sites which react to form di-PEGylated molecules and so on, resulting in a mixture of products differing both in the number of PEG groups attached and their attachment positions.

This method results not only in poor yields of the product of interest but requires further purification steps in order to obtain a homogenous end product.

Where the activated polymer and/or by-products are toxic, biological assays of the desired end product cannot be performed without extensive, time-consuming and costly purification steps.

A wide range of separation techniques have been used to separate PEGylated products and it is anticipated that these would be known to a skilled addressee.

However, the heterogenerity and the dose similarity of physicochemical properties between products makes the purification of PEGylated products difficult.

Gel filtration can be used, but determination of molecular weight by this method is not reliable as the media cannot be readily calibrated.

PEGylated molecules show different elution times from compounds with the same molecular weight, as PEG groups typically occupy approximately twice the volume as a similar sized protein. Molecular weights cannot thus be accurately determined, making accurate separation and identification difficult.

Further, while it is possible to ignore the effect on the active site during conjugation and rely on the later separation of the heterogeneous mixture, the ability to control the PEGylation reaction would simplify the downstream processing required to obtain a homogenous end product.

A number of techniques have been used with varying success to attempt to control the PEGylation reaction by limiting the number of active sites available for conjugation.

The simplest and most obvious is to carry out conjugation in the presence of a substrate or inhibitor that binds to the active site. Some protection can be afforded by this method but, particularly where the binding molecule is small, PEGylation of nearby sites is not necessarily prevented.

Another method involves the use of a “solid-phase” reaction, described by Monkarsh et al, 1997. In this method, the native protein is bound to a chromatographic resin (usually an ion exchange media) and activated PEG is recirculated through the column. After a given time the activated PEG feed is stopped and the resin is washed.

Differences in the strength of electrostatic interaction are then exploited to elute the PEGylated products before more native protein is then loaded, and the activated PEG is again introduced to the column, to repeat the process.

Unfortunately, this method yields little, if any, difference in the degree of product heterogeneity from the liquid-phase method.

Such processes are also typically difficult to control. As such, the inability to reduce the levels of product heterogeneity formed during PEGylation reactions necessitates high purification costs which offset the benefits of PEGylated products.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be dearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a reaction process,

characterised by the step of

introducing reaction components to a medium,

wherein at least one of the reaction components has a different flow rate from the other reaction component(s) through the medium, so that a moving reaction phase is formed which produces reaction products.

According to another aspect of the present invention there is provided a reaction process of bringing reaction components together in a moving reaction phase through a medium,

characterised by the further step of

separating the reaction products from the medium.

The term “reaction component” can be defined as any compound which takes part in the reaction and which exhibits different flow characteristics through a medium from the other reaction components.

The reaction component may be any compound which reacts with another reaction component to form a different compound. However, this should not be seen as a limitation on the present invention in any way as other reaction components could include catalysts, which are neither consumed nor altered by the chemical reaction in which it participates; or buffer components; which control pH and thus the moving reaction phase.

In some embodiments the difference in flow rate through the medium will be a consequence of the molecular size of at least one reaction component.

However, differences in flow rate may be due to other physicochemical characteristics such as electrostatic charge, ligand interaction and so forth. As such, this should not be seen as a limitation on the present invention in any way.

A number of the reaction components may have the same flow rate characteristics, the moving reaction phase being determined by another reaction component with a different flow rate.

The term “moving reaction phase” can be defined as the point where all reaction components necessary for a given reaction are present in the same space in the medium at the same period. In time as dictated by the differing flow characteristics of at least some of the reaction components.

The physical and/or chemical properties of the medium which provide differing flow rates to the reaction components naturally leads to separation of the reaction products through the same principles.

The term “medium” can be defined as anything which imparts different flow rates on the basis of a compound's physicochemical properties. The medium may be a chromatographic resin, porous beads, gel, viscous solvent or so forth.

In preferred embodiments of the present invention, the medium will preferably be porous beads, with the moving reaction phase and separation occurring through the use of size exclusion chromatography, or gel filtration. However, this should not be seen as a limitation on the present invention in any way.

The beads are preferably made of a crossed linked polymeric material such as dextran or agarose. Molecules with a molecular weight larger than the pores in the beads are excluded, moving quickly through the column whereas the movement of smaller molecules which enter the pores is retarded.

The moving reaction zone may be formed through several mechanisms.

Differences in molecular size between reaction components and products allows the control of the moving reaction phase by altering the physical and/or chemical properties of the medium.

For example, in some preferred embodiments the moving reaction phase may be controlled by the molecular weight of buffer components.

The reaction components may be mixed together in a low pH buffer to prevent a reaction, and then injected on to a chromatographic column that has been equilibrated at a high pH, so that the reaction components will move into a high pH buffer early in the column and react. The moving reaction zone is not formed by the difference in migration rate between the reaction components and the low molecular weight buffer molecules.

Reaction time can also be controlled by adjusting the volumes of the reaction components and the overall flow rate through the column.

Differences in molecular size between the reaction components and the reaction products may also result in a rate of progress of the products through the column that differs from that of the moving reaction zone.

The products can therefore be selectively removed from the reaction zone, preventing them from being involved in subsequent reactions.

In some media, the inventors have found that the pore size may prevent particular chemical products from forming, perhaps through steric hindrance. For certain reactions where the reaction components may have multiple active sites, this reaction process may be used to produce a dominant reaction product of a particular size.

It is anticipated that the properties of the medium may also be selected to affect the orientation of the reaction components such that some selectivity of active site may be possible in reactions where multiple active sites exist.

Additionally, some active sites may be protected through the use of protection chemistry as known in the art to selectively produce a particular chemical species.

The use of differing flow rates through a media combines both reaction and separation into a single unit operation. By controlling the volumes of reaction components used, the flow rate through the column and by choosing the appropriate media and length of column, separation of reaction products and reaction components can occur.

The ability to produce a specific compound and separate this from other products and reaction components enables compounds to be formed with a very high yield and purity.

In preferred embodiments of the present invention, the technique will be used for protein PEGylation.

However, this should not be seen as a limitation as the technique may extend to any process in which reaction product(s) differ in molecular size from the reaction component(s), such as glycosylation reactions, polymerisation, cleavage reactions and so forth.

It is anticipated a skilled addressee using minor experimentation would be able to match an appropriate medium with the separation range required for the substances of interest, allowing the technique to be used for a wide range of compounds and reactions.

Protein PEGylation is a technique wherein the circulation half lives of small therapeutic proteins (<20 kDa) can be dramatically improved by increasing their molecular size through the covalent attachment of one or more polyethylene glycol (PEG) groups.

The attachment of PEG groups not only prevents early glomerular filtration and excretion, but the water of hydration surrounding each PEG group moves constantly and is thought to slow immunological attack via steric hindrance.

Increasing the circulation half rife of a therapeutic protein has the dual benefits of increasing its overall efficacy and reducing the required frequency of dosage. The latter aspect can be an important factor. In reducing patient discomfort and increasing product acceptability; particularly for treatments requiring repeated intravenous or subcutaneous administration.

However, standard PEGylation reaction processes not only give poor yields but result in product heterogeneity to a degree which is unacceptable in a therapeutic protein, necessitating further processing to obtain a homogeneous end product.

Downstream processing is difficult because the physicochemical properties of the PEGylated protein species are very similar, apart from differences in their effective molecular size, and each step required for downstream processing decreases overall yield.

The low yields from PEGylation and the need for downstream processing represent significant costs for the PEGylated product over and above its native form.

Given that the native therapeutic protein (generally a recombinant protein) must be purified to homogeneity prior to PEGylation, the overall production cost of a PEGylated protein is likely to be very high, offsetting the potential benefits of PEGylation.

Using size-exclusion chromatography, the inventors have found a reaction process to control the PEGylation reaction and allow the substantially simultaneous purification of the products, which they have termed size-exclusion reaction chromatography (SERC).

The SERC techniques provides a range of advantages that overcome many of the problems associated with the prior art.

SERC allows molecules to be partitioned within the mobile phase according to their molecular size and shapes and the pore size distribution of the stationary phase.

A difference in molecular size between the reaction products and either of the reaction components results in a rate of progress of the products through the column that differs from that of the reaction zone.

Products can therefore be removed selectively from the reaction zone, preventing them from being involved in subsequent reactions, limiting over-reaction.

Further, the inventors have also found that certain size exclusion media can prevent the formation of a given product that exceeds the size of the pore in which the reaction components are contained.

By using a size exclusion media wherein the reaction components are poorly separated but in which the pore size excludes products larger than the target PEG-protein size, it is anticipated that a native protein could be converted to a specified final molecular size, eliminating the need for further purification and producing a substantially homogenous end product at very high yield.

For reactions such as protein PEGylation, in which successive addition of PEG groups to the protein results in a significant molecular size increase, this presents a reaction process by which a dominant final PEG-protein size can be produced.

It is also anticipated that the shape of the pore in the reaction components may affect the orientation of the compounds during conjugation, such that some selectivity of active site may be possible in reactions where multiple active sites exist.

SERC thus allows a transient, moving reaction zone to be formed within a size exclusion chromatography column which can control the time of contact between reaction components, selectively remove products from the reaction zone and selectively inhibit reactions based on molecular size.

The ability to control not only the partitioning of molecular species with different molecular weights through a column, but also the use of a medium which prevents the formation of a product larger than the pore size, has huge potential for controlling a wide range of chemical reactions.

By controlling the pore sizes and the pore size distribution correctly, it should be possible to create a size exclusion medium that will prevent reactions between activated PEG molecules (or any other molecules) and molecules of a specific size, allowing size exclusion mediums to be designed specifically for the production of a specified chemical species of interest.

The advantages of the use of this technology in relation to PEGylation chemistry alone are enormous given the potential benefits of PEGylated products, which previously have suffered due to a number of serious disadvantages in the prior art.

An example of how the present invention can be used in relation to PEGylation is given below. However, this example is meant to be illustrative only and the present invention is not limited to same.

In this example, a short pulse of activated PEG is injected onto a size-exclusion column running at a constant volumetric flow rate.

A subsequent pulse of native protein eventually catches up to the slow-moving PEG pulse, forming a transient, moving reaction zone that exists for as long as the two reaction component pulses occupy the same axial position in the column.

The addition of a 5000 Da PEG group adds significantly more than 5000 Da to the effective molecular weight of the protein, as measured by size-exclusion chromatography, due to the water of hydration associated with the PEG group.

The addition of a single 5000 Da PEG group to a 35.8 kDa protein results in a mono-PEGylated protein with an apparent molecular weight in excess of 100 kDa. Thus the PEGylated product is partitioned differently from either of the two reaction components.

If the size-exclusion medium is chosen carefully, the PEGylated product will be partitioned into the void space between the stationary phase and so will move relatively quickly out of the reaction zone. Additional column length downstream of the reaction zone allows separation of the reaction components and products.

By choosing appropriate reaction component pulse volumes, reaction component concentrations and volumetric flow rates, a large degree of control can be exercised over the reaction conditions and the length of the subsequent separation zone.

The present invention also includes methods for controlling chemical reactions through the formation of a moving reaction phase, kit sets and media therefore; and reaction products formed by such reactions.

### DESCRIPTION OF THE DRAWINGS

The invention will be further described by reference to the figures of the accompanying drawings in which:

FIG. 1 Schematic of the change in individual PEGylation product concentrations with time during batch PEGylation.

FIG. 2 Elution profiles of native α-lactalbumin and β-lactoglobulin on a Superdex 200 HR 10/30 size-exclusion column.

FIG. 3 The effect of reaction time on the apparent molecular weight of α-lactalbumin for batch PEGylation.

FIG. 4 The effect of reaction time on the apparent molecular weight of lactoglobulin for batch PEGylation.

FIG. 5 The elution profile for SERC PEGylation of α-lactalbumin (UV absorbance at 280 nm).

FIG. 6 The elution profile for SERC PEGylation of β-lactoglobulin (UV absorbance at 280 nm).

FIG. 7 Molecular weight profiles of fractions collected during SERC PEGylation of α-lactalbumin. Chromatograms are stacked to aid readability. Numbers refer to fractions in FIG. 5.

FIG. 8 Molecular weight profiles of fractions collected during SERC PEGylation of β-lactoglobulin. Chromatograms are stacked to aid readability. Numbers refer to fractions in FIG. 6.

FIG. 9 Molecular weight profiles of pooled protein fractions collected during SERC PEGylation of α-lactalbumin.

FIG. 10 Molecular weight profiles of pooled protein fractions collected during SERC PEGylation of β-lactoglobulin.

FIG. 11 Dependence on the apparent molecular weight of the number of PEG groups attached.

FIG. 12 Diagrammatic representation of the moving reaction phase formed between two reaction components using size-exclusion reaction chromatography.

FIG. 13 Shows the results of modelling the SERC process for PEG and protein migrating at different rates.

FIG. 14 Shows the results of modelling wherein the media does not separate the two reaction components, but sharply excludes any PEGylation species.

### EXAMPLE 1

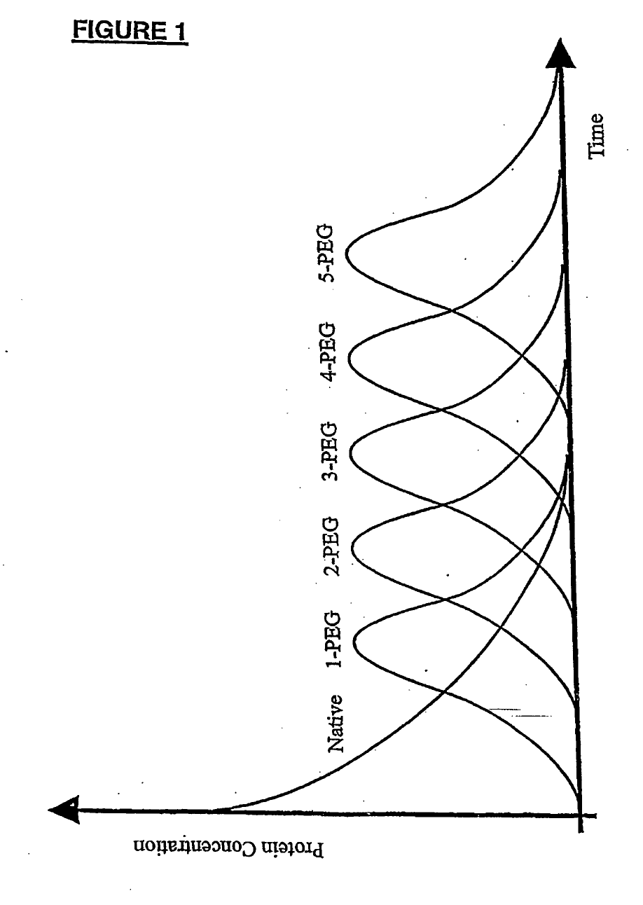

SERC PEGylation was conducted on two model proteins, α-lactalbumin (14.2 kDa) and β-lactoglobulin (a 35.8 kDa dimmer), and compared with standard batch PEGylation techniques. A diagram showing the change in individual PEGylation product concentrations over time during batch PEGylation is shown in FIG. 1.

It was anticipated that the large molecular weight products of PEGylation would be partitioned into the void volume of the size-exclusion media and therefore be selectively removed from the slower-moving reaction zone, limiting the extent of the reaction and minimising multiple PEGylation reactions.

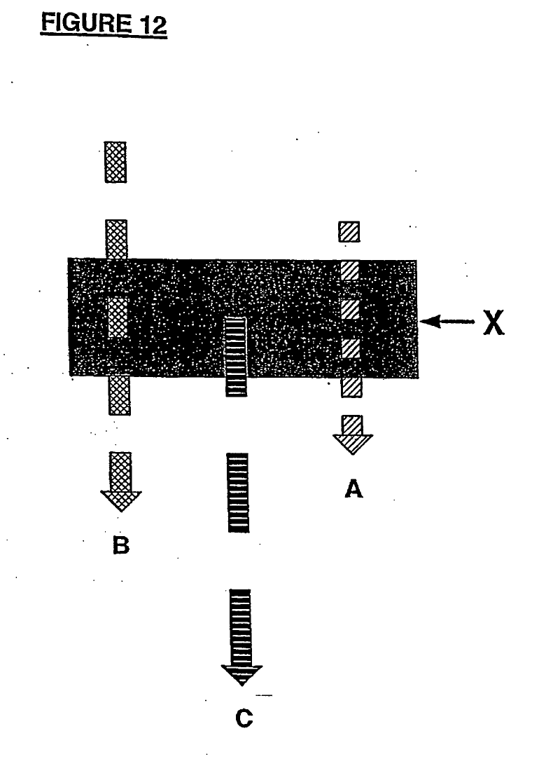

A diagrammatic representation of the moving reaction zone formed between the reaction components using size-exclusion reaction chromatography is shown by FIG. 12.

In this figure, the faster moving protein (B) catches up with the slower moving protein (A) to form a moving reaction zone (X) that exists as long as the two reaction components are in the same space of the medium, at the same time.

The reaction product (C) by virtue of its larger apparent molecular weight is moving quickly out of the reaction zone.

In the course of experiments it was also discovered that the size-exclusion medium also acts to prevent the formation of a product larger than the pore size in which the reaction components were located.

It is concluded that this latter effect has far greater potential for controlling such reactions, through appropriate choice of the size-exclusion media pore size, than differences in migration rates through the column alone.

Materials & Methods

Reagents

Activated PEG reagent, mPEG succinimidyl propionate (mPEG-SPA), molecular weight 5000 Da, was purchased from Shearwater Corporation, Alabama, α-lactalbumin, (14,200 Da) and β-lactoglobulin dimer (35,800 Da) were purchased from Sigma-Aldrich Corporation, Australia.

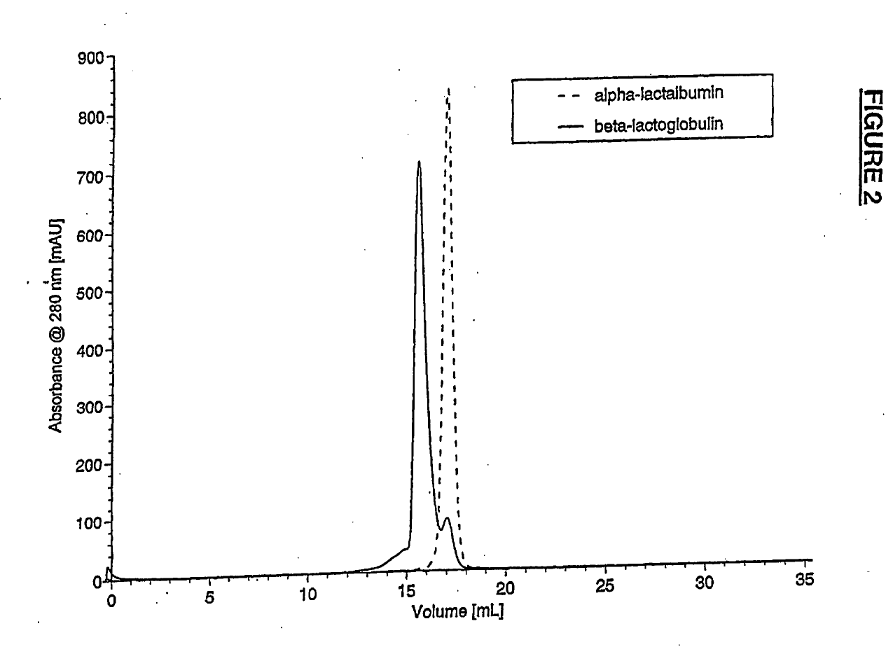

Gel filtration chromatograms of the two proteins are shown in FIG. 2. A minor amount of α-lactalbumin is evident in the β-lactoglobulin material but otherwise the two proteins are sufficiently pure for PEGylation studies.

All solutions were made up in 20 mM Tris-HCl buffer at pH 7.5.

Batch PEGylation

Batch PEGylation was carried out with each protein individually to follow the reaction progress in the absence of the size-exclusion medium.

10 mL of 20 mg/mL mPEG-SPA solution was added to 10 mL of 20 mg/mL native protein and stirred in an open 25 mL beaker at room temperature. One sample (approx. 1 mL) was withdrawn immediately after mixing the reaction components and further samples were taken at 10-minute intervals for 1 hour.

Samples were placed into vials and acidified with one drop of 1N HCl to stop the reaction.

SERC PEGylation

SERC PEGylation of each protein was carried out to determine the elution profile from the size-exclusion column and to compare the overall product profiles with the corresponding batch PEGylation results.

A HiLoad 16/60 Superdex 75 pg size-exclusion column (Amersham Pharmacia Biotech, Uppsala, Sweden) was connected to an AKTAexplorer 10XT liquid chromatography system (Amersham Pharmacia Biotech, Uppsala, Sweden) and equilibrated with two column volumes of 20 mM Tris-HCl at pH 7.5.

A specified volume of mPEG-SPA (10 mg/mL) was injected onto the column, followed immediately by an equal volume of native protein (10 mg/mL). UV absorbance was read at 280 nm.

5 mL fractions were collected continuously from the outlet stream after an initial 40 mL of buffer (i.e. slightly less than the void volume of the column) had passed through the column.

1 mL samples of each fraction was withdrawn by pipette and placed into auto sampler vials for size-exclusion analysis. The remaining portions of those fractions that contained proteins (identified from previous runs) were pooled and a sample of the pool was taken for size-exclusion analysis.

Reaction component injection volumes and flow rates were then set to give estimated reaction zone residence times (i.e. the times for which the reaction components overlapped in the column) by assuming that each reaction component progressed through the column at a constant velocity, related to its Kₐᵥ value (see Appendix 1).

Analysis

40 μL samples were injected onto a Superdex 200 HR10/30 size-exclusion column (Amersham Pharmacia Biotech, Uppsala, Sweden) connected to an AKTAexplorer 10XT system (Amersham Pharmacia Biotech, Uppsala, Sweden) using an Autosampler A900, running phosphate buffered saline at 0.5 mL/min.

The size-exclusion column was first calibrated using standard molecular weight markers (Amersham Pharmacia Biotech, Uppsala, Sweden). UV absorbance was read at 280 nm and the molecular weight profiles of samples recorded.

Individual samples of α-lactalbumin and β-lactoglobulin (dimer) and mPEG-SPA all gave retention volumes consistent with their native molecular weights.

Results

Batch PEGylation

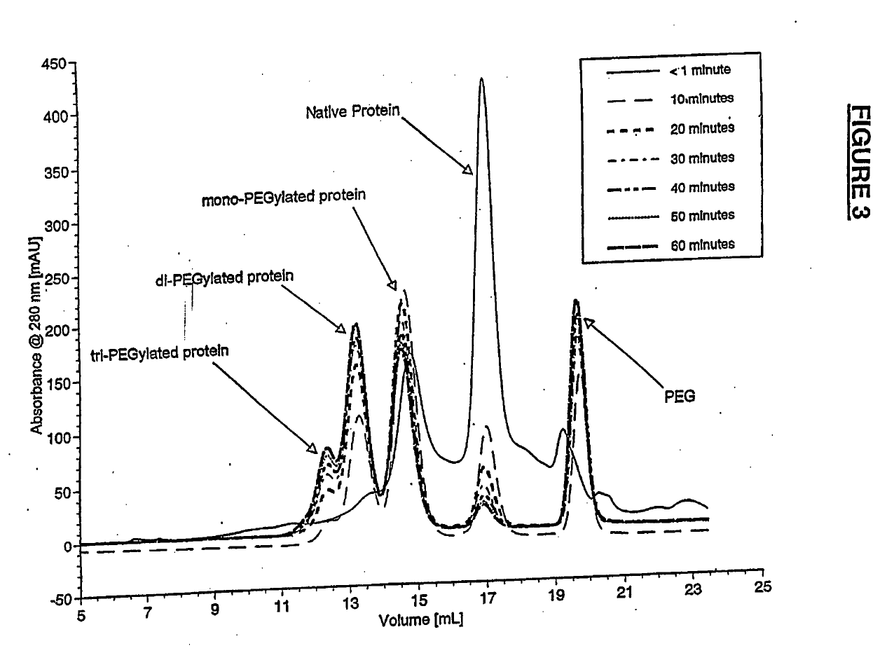

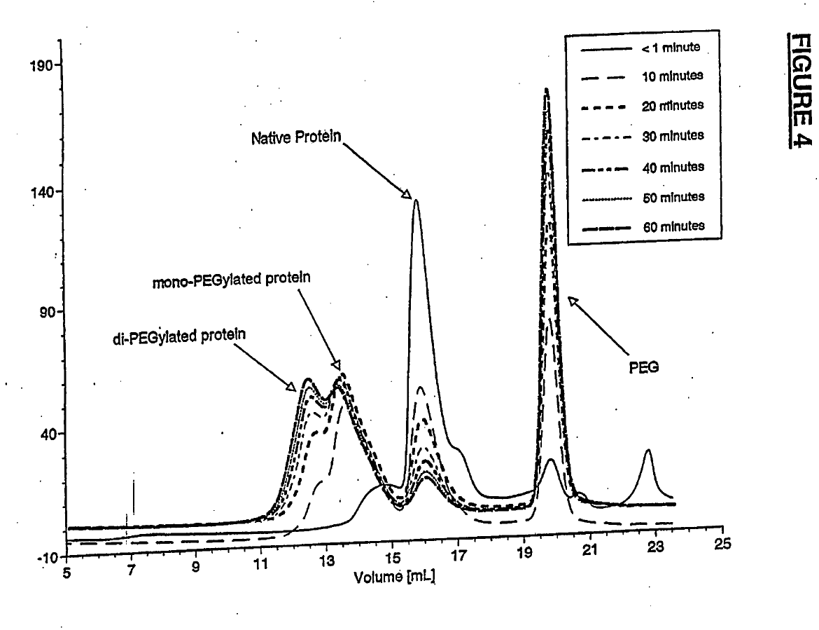

FIGS. 3 and 4 show gel filtration chromatograms for the products of batch PEGylation of α-lactalbumin and β-lactoglobulin, respectively. In each case there is a shift in the peak areas with time, showing a reduction in the native protein peak, an initial increase and then decrease in the mono-PEGylated protein peak, and corresponding increases in the multiple-PEGylated species with time.

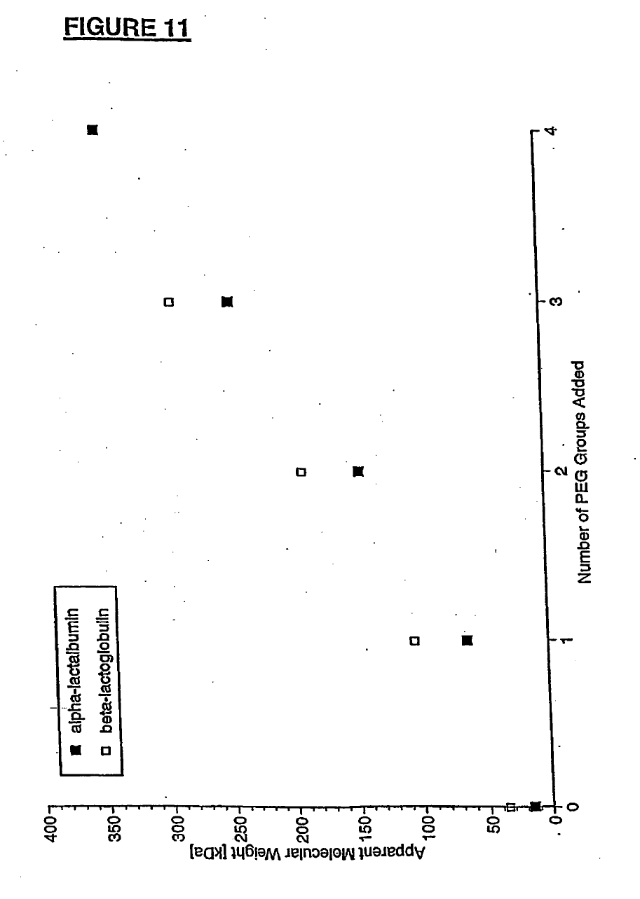

FIG. 11 shows the apparent molecular weights of the PEGylation products for both α-lactalbumin and β-lactoglobulin as a function of the number of PEG groups assumed to have been attached. The curves in FIG. 11 show that the increase in apparent molecular weight resulting from the addition of successive PEG groups is consistent between these proteins, with each 5 kDa PEG group adding between 50 and 100 kDa to the effective molecular weight of the proteins. Notably, when analysed separately, mPEG-SPA had a retention volume on the analytical size-exclusion column that is consistent with its 5 kDa molecular weight (data not shown).

SERC PEGylation

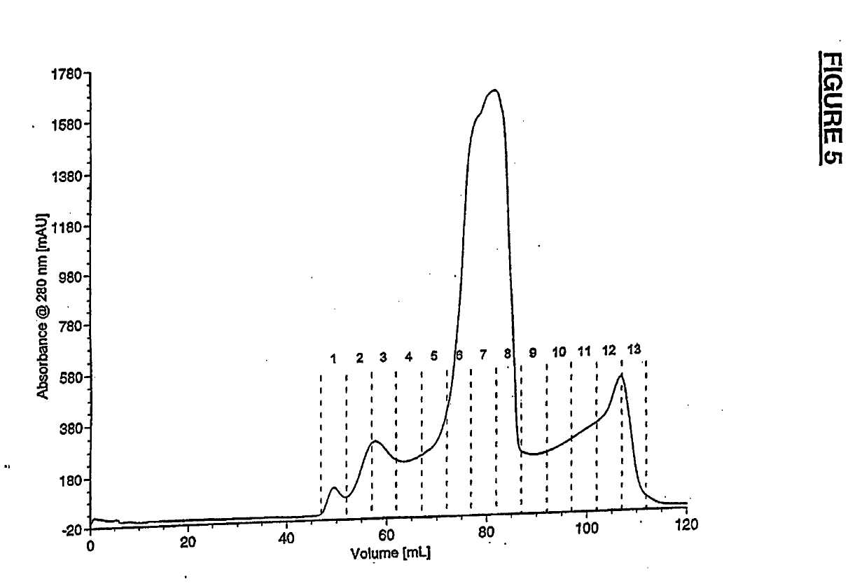

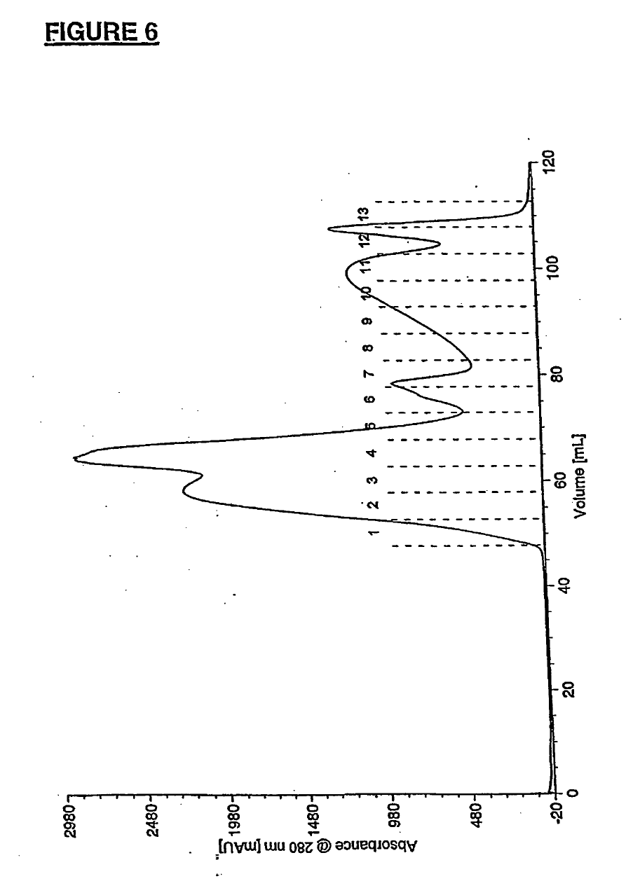

FIGS. 5 and 6 show the elution profiles obtained from the SERC process for α-lactalbumin and β-lactoglobulin, respectively.

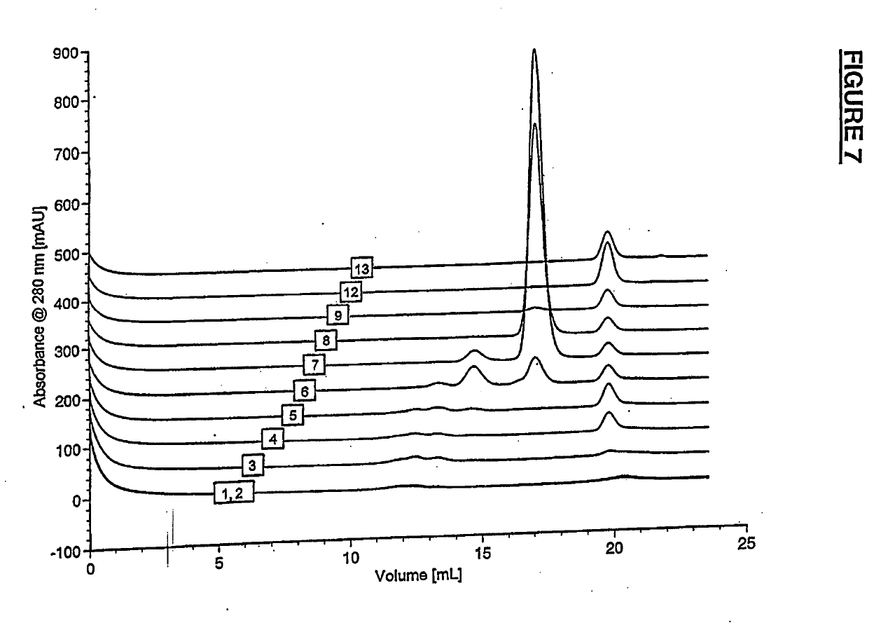

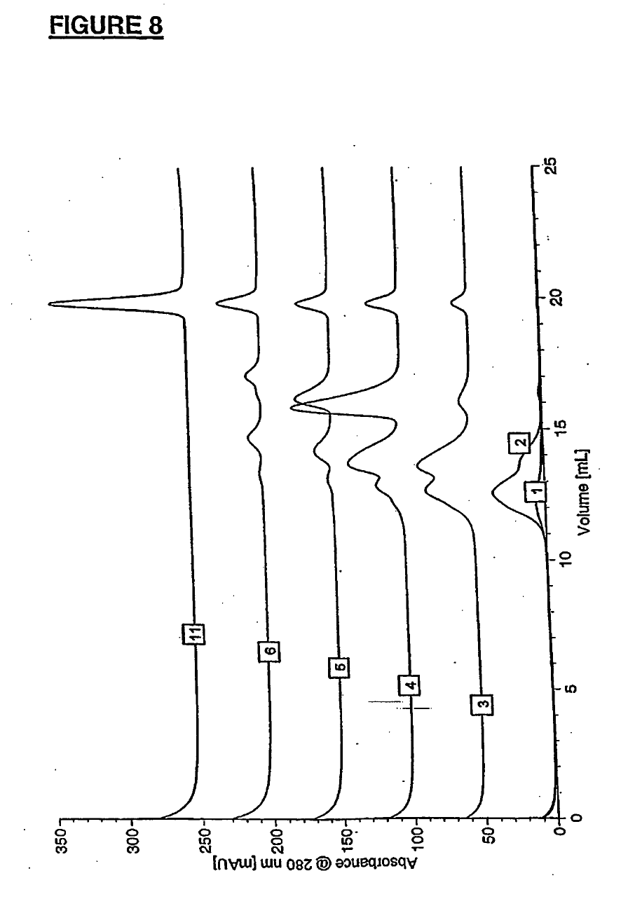

Gel filtration analyses of the fractions collected are given in FIGS. 7 and 8. These show that the larger molecular weight species were eluted early and that there was good separation of the protein species from the residual mPEG-SPA and other low molecular weight reaction products. Thus substantially simultaneous reaction and separation was achieved.

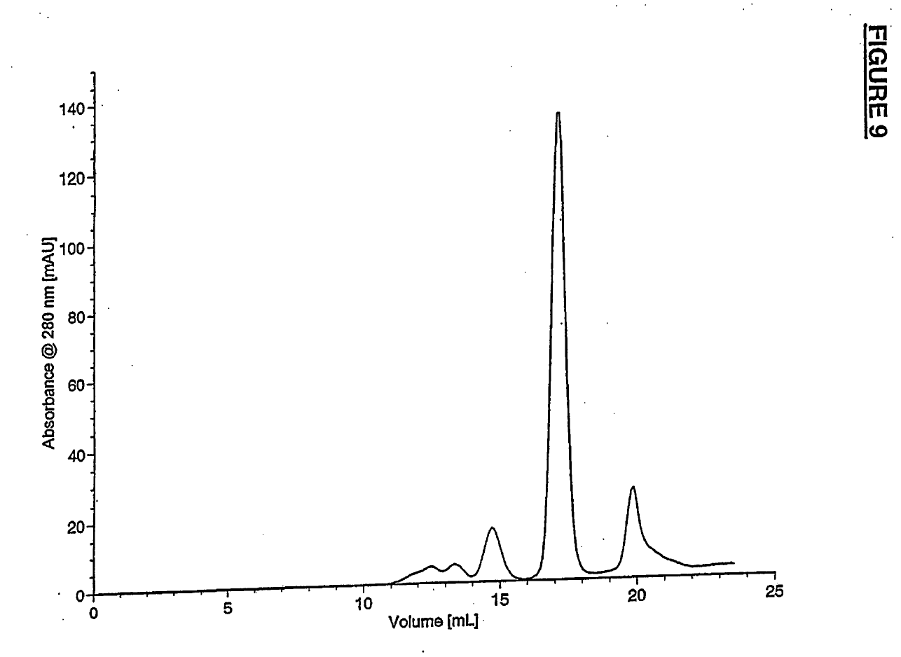

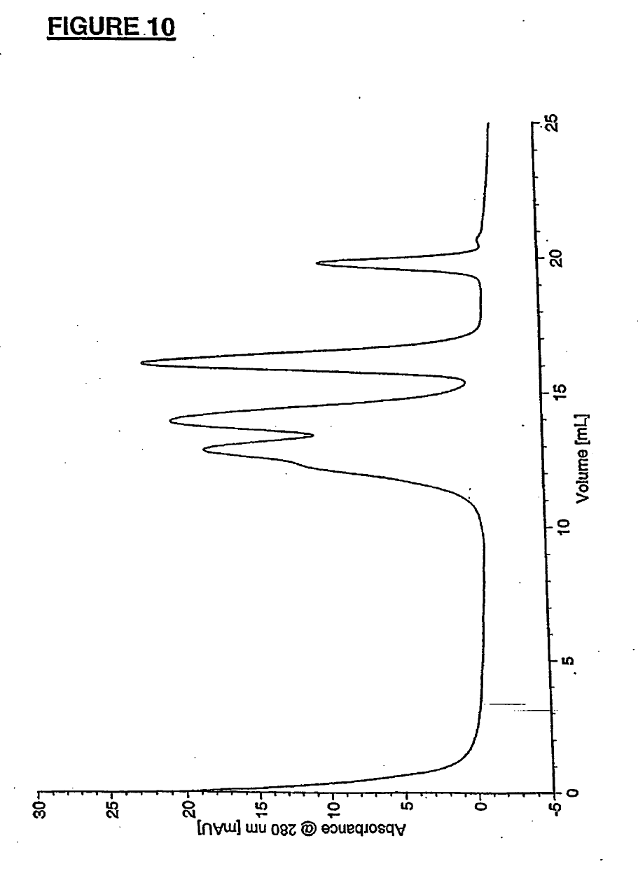

FIGS. 9 and 10 show the pooled protein fractions eluted from the SERC process for a α-lactalbumin and β-lactoglobulin, respectively.

FIG. 9 shows that the SERC process has resulted in a significantly reduced extent of reaction of α-lactalbumin than the corresponding batch process (FIG. 3).

Comparison of FIG. 10 (SERC) with FIG. 4 (batch) shows that there is a slight difference in the molecular weight profiles resulting from the two reaction reaction processes for β-lactoglobulin, but that the reaction is not inhibited to the extent that it is for α-lactalbumin.

Discussion

The SERC PEGylation of β-lactoglobulin with a calculated residence time of 34 minutes resulted in a higher ratio of mono-PEGylated product than the corresponding batch process at 30 minutes. This is consistent with the hypothesis that the larger, PEGylated molecules are accelerated out of the reaction zone at a faster rate than the native protein, thereby reducing the probability that they will be involved in further PEGylation.

The results show that protein PEGylation can be carried out in a size-exclusion column and that reaction components and products are separated in the process.

Size-exclusion is a logical choice for the purification of PEGylated proteins and this combination of reaction and separation in a single unit operation should reduce capital costs and eliminate the need for handling and conditioning between reaction and separation steps.

Further, SERC PEGylation of α-lactalbumin (FIG. 9) resulted in a significantly lower extent of reaction than batch PEGylation (FIG. 3), the dominant product being native α-lactalbumin. An explanation for this is that α-lactalbumin is mainly partitioned into pores that prevent the simultaneous occupancy by mPEG-SPA molecules in an orientation suitable for reaction, even though both molecules should be able to penetrate the same pores.

This is in contrast with the result for β-lactoglobulin, where the degree of reaction inhibition is markedly less (FIG. 10). β-lactoglobulin, though larger than α-lactalbumin, is apparently partitioned in such a way as to remain largely accessible by mPEG-SPA molecules with sufficient mobility to react.

If there were no steric hindrance effects by the pores then one might-expect β-lactoglobulin to react about 24% faster than α-lactalbumin, based on the batch reaction results. However, 72.5% of the β-lactoglobulin reacted in the SERC column with a 34-minute reaction zone residence time, compared with only 16.7% for α-lactalbumin with a 55-minute reaction zone residence time.

This is not explained by differences in concentration due to partitioning effects alone. It is therefore likely that steric hindrance due to the pores is an important factor and that the α-lactalbumin molecules fit tightly into the pores they occupy whereas β-lactoglobulin molecules fit loosely into the pores they occupy.

This finding suggests a reaction process for controlling reactions by controlling the pore-sizes and pore-size distribution so as to obtain a high yield of a product with a specific molecular weight.

A size-exclusion medium that separates the reaction components poorly but that sharply excludes products larger than the target PEGylated protein size would maximise the reaction time but minimise the formation of over-PEGylated products.

This would allow a size-exclusion medium to be designed specifically for the production of, for example, a di-PEGylated protein. The SERC process could then be run with an excess of activated PEG, allowing conversion of native and mono-PEGylated protein species to di-PEGylated protein to be maximised, while inhibiting further PEGylation through steric hindrance.

The by-products and residual PEG species could be separated from the di-PEGylated protein product by collecting fractions in the exit stream, producing a largely homogeneous product at high yield.

This, in combination with a reduction in capital costs and eliminating handling between reaction and separation steps, has the potential to greatly improve process economics.

### EXAMPLE 2

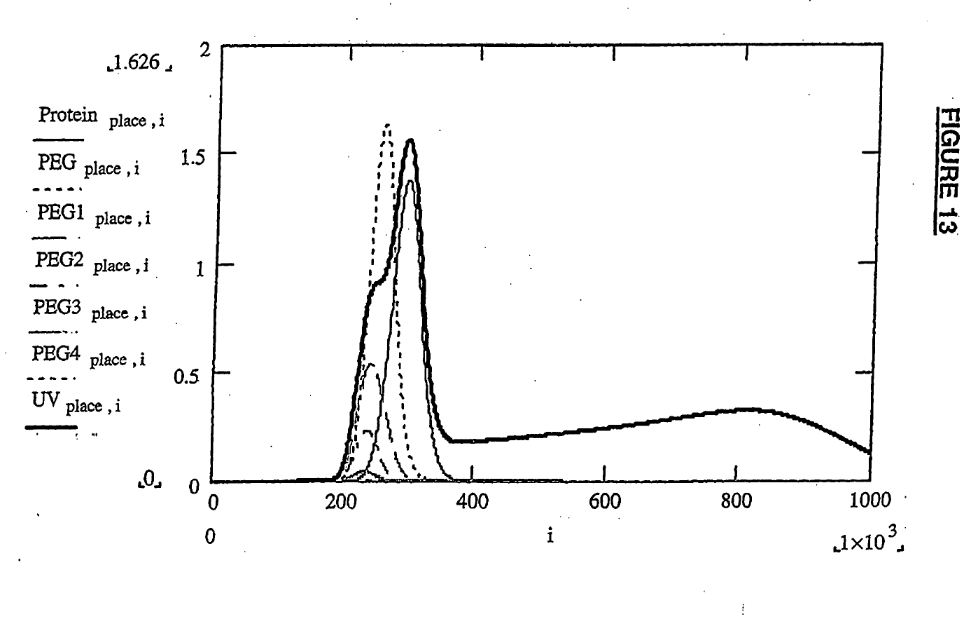

FIG. 13 shows the results of modelling the SERC process for the case where PEG and protein migrate at differing rates through the column and all PEGylated products are excluded into the void space.

The solid black line is the UV absorbance trace expects, which is made up of the sum of the UV-active species in the column. Note the peaks at the left hand side, which show mono-, di-, tri- and tetra-PEGylated products occur, and add up to give the overall UV absorbance. To the right is a broad curve of low molecular weight by products of the reaction.

The modeled curve in FIG. 13 corresponds roughly to the results of the experimental work, shown in FIG. 6. There is a reasonable, though not perfect, match in trends which provides initial confidence in the validity of the model.

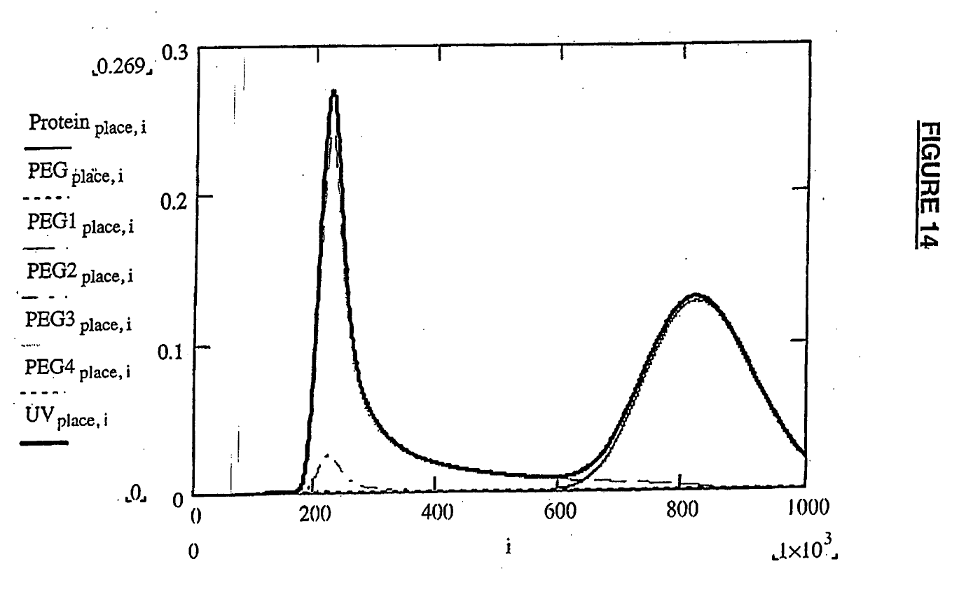

FIG. 14 shows an extreme case where a custom made size exclusion media is available which does not separate the two reactants (which are injected together) but sharply excludes any PEGylated species.

In this case, on a practical level, one would have to mix the reactants together in a low pH buffer (for example pH 5.5) to prevent reaction, and then inject them onto column that has been equilibrated at a high pH (for example pH 8) so that the reactants will move into a high pH buffer early in the column and react. Thus the moving reaction zone is formed by the difference in migration rates between the reactants (PEG and protein) and the low molecular weight buffer molecules.

The result shows that under these conditions, the reaction product is almost totally mono-PEGylated, with only a small amount of di-PEGylated protein present.

Aspects of the present invention have been described by way of example only and it should be appreciated that modifications and additions may be made thereto without departing from the scope thereof as defined in the appended claims.

1. A reaction process, comprising introducing reaction components to a medium, wherein at least one of the reaction components has a different flow rate from the other reaction component(s) through the medium, so that a moving reaction phase is formed which causes reaction products.

2. The reaction process as claimed in claim 1 further comprising separating the reaction products from the medium.

3. The reaction process as claimed in claim 1 or claim 2 wherein the differences in flow rate through the medium are a consequence of a molecular weight of at least one reaction component.

4. The reaction process as claimed in claim 1 or claim 2 wherein the differences in flow rate through the medium are due to electrostatic charge.

5. The reaction process as claimed in claim 1 or claim 2 wherein the differences in flow rate through the medium are due to ligand interaction.

6. The reaction process as claimed in claim 1 wherein the properties of the medium that provide differing flow rates to the reaction components also leads to separation of the reaction products.

7. The reaction process as claimed in claim 1 wherein the medium includes porous beads.

8. The reaction process as claimed in claim 7 wherein the beads are made of a cross-linked polymeric material.

9. The reaction process as claimed in claim 8 wherein the cross-linked polymeric material includes dextran.

10. The reaction process as claimed in claim 8 wherein the cross-linked polymeric material includes agarose.

11. The reaction process as claimed in claim 1 wherein the moving reaction phase is controlled by altering the properties of the medium.

12. The reaction process as claimed in claim 1 wherein the moving reaction phase is controlled by altering the volumes of the reaction components through a column.

13. The reaction process as claimed in claim 1 wherein the moving reaction phase is controlled by altering the overall flow rate through a column.

14. The reaction process as claimed in claim 1 wherein the reaction products are selectively removed from a reaction zone, preventing them from being involved in subsequent reactions.

15. The reaction process as claimed in claim 14 wherein the reaction products are selectively removed from the reaction zone due to differences in molecular size between the reaction components and the reaction products.

16. The reaction process as claimed in claim 1 wherein properties of the medium are selected to substantially prevent particular reaction products from forming.

17. The reaction process as claimed in claim 16 wherein the properties of the medium are selected to orient the reaction components to provide selectivity of an active site in reactions where multiple active sites exist.

18. The reaction process as claimed in claim 17 wherein said active site is protected through the use of protection chemistry to selectively produce a particular reaction product.

19. The reaction process as claimed in claim 1 wherein the reaction process is used for glycosylation reactions.

20. The reaction process as claimed in claim 1 wherein the reaction process is used for polymerisation.

21. The reaction process as claimed in claim 1 wherein the reaction process is used for cleavage reactions.

22. The reaction process as claimed in claim 1 wherein the reaction process is used for protein PEGylation.

23. The reaction process as claimed in claim 1 wherein the reaction process is controlled using size-exclusion reaction chromatography.

24. A method of protein PEGylation, comprising forming a moving reaction phase using a reaction process as claimed in claim 1.

25. Reaction products produced by the reaction process of claim 1.

26. The reaction products as claimed in claim 25 wherein the products are PEGylated protein.

27. (canceled)

28. A kitset to bring two or more reaction components together, comprising
at least two reaction components, and
a medium,
wherein at least one of the reaction components has a different flow rate from the other reaction component(s) through the medium, so that a moving reaction phase is formed, which produces reaction products.

29. A kitset for use in a reaction process as claimed in claim 1, comprising
unit volumes of at least two reaction components,
a medium,
instructions and
means for bringing two or more reaction components together in a moving reaction phase.

30. (canceled)

31. (canceled)

32. (canceled)
